(19) United States
(12) Patent Application Publication
(10) Pub. No.: US 2024/0418979 A1
(43) Pub. Date: Dec. 19, 2024

(54) DRIVE DEVICE, ENDOSCOPE SYSTEM, AND DRIVE METHOD

(71) Applicant: OLYMPUS MEDICAL SYSTEMS CORP., Tokyo (JP)

(72) Inventors: Yuji SAKAKI, Tokyo (JP); Kota OGAWA, Tokyo (JP)

(73) Assignee: OLYMPUS MEDICAL SYSTEMS CORP., Tokyo (JP)

(21) Appl. No.: 18/817,756

(22) Filed: Aug. 28, 2024

Related U.S. Application Data
(63) Continuation of application No. PCT/JP2022/009558, filed on Mar. 4, 2022.

(51) Int. Cl. G02B 23/24; G02B 7/08

(52) U.S. Cl. CPC G02B 23/243; G02B 7/08

(57) ABSTRACT

A drive device includes: a fixed portion; a movable portion that is provided slidably inside the fixed portion, the movable portion being configured to hold a lens; a drive portion configured to cause the movable portion to slide along an optical axis of the lens with respect to the fixed portion; a driver configured to supply a current to the drive portion; and a controller configured to control the driver, the controller including a processor including hardware, the processor being configured to cause the driver to supply a first current that is small to the drive portion and cause the driver to repeatedly supply a second current larger than the first current on a predetermined cycle, and a direction of movement of the movable portion by the first current or the second current being a same in the predetermined cycle.

### CROSS-REFERENCE TO RELATED APPLICATION

This application is a continuation of International Application No. PCT/JP2022/009558, filed on Mar. 4, 2022, the entire contents of which are incorporated herein by reference.

### BACKGROUND

### 1. Technical Field

The present disclosure relates to a drive device, an endoscope system, and a drive method.

### 2. Related Art

In a known technique for stopping a lens in a lens drive device that moves the lens along an optical axis by driving of a stepping motor, the exciting current required for the stopping is reduced after the lens has been moved to an excited stable position (see, for example, Japanese Patent Application Laid-open No. 2011-197163).

### SUMMARY

In some embodiments, a drive device includes: a fixed portion; a movable portion that is provided slidably inside the fixed portion, the movable portion being configured to hold a lens; a drive portion configured to cause the movable portion to slide along an optical axis of the lens with respect to the fixed portion; a driver configured to supply a current to the drive portion; and a controller configured to control the driver, the controller including a processor including hardware, the processor being configured to cause the driver to supply a first current that is small to the drive portion and cause the driver to repeatedly supply a second current larger than the first current on a predetermined cycle, and a direction of movement of the movable portion by the first current or the second current being a same in the predetermined cycle.

In some embodiments, an endoscope system includes: an endoscope including an optical unit, the endoscope being configured to be inserted inside a subject to observe an interior of the subject; and a control device to which the endoscope is detachably connected, the control device being configured to control driving of the optical unit, the optical unit including: a fixed portion; a movable portion that is provided slidably inside the fixed portion, the movable portion being configured to hold a lens; and a drive portion configured to cause the movable portion to slide along an optical axis of the lens with respect to the fixed portion, the control device includes: a driver configured to supply a current to the drive portion; and a controller configured to control the driver, the controller including a processor including hardware, the processor being configured to cause the driver to supply a first current that is small to the drive portion and cause the driver to repeatedly supply a second current larger than the first current on a predetermined cycle, and a direction of movement of the movable portion by the first current or the second current being a same in the predetermined cycle.

In some embodiments, provided is a drive method executed by a drive device including: a fixed portion; a movable portion that is provided slidably inside the fixed portion, the movable portion being configured to hold a lens; a drive portion configured to cause the movable portion to slide along an optical axis of the lens with respect to the fixed portion; and a driver configured to supply a current to the drive portion. The drive method includes: causing the driver to supply a first current that is small to the drive portion and causing the driver to repeatedly supply a second current larger than the first current on a predetermined cycle, and a direction of movement of the movable portion by the first current or the second current being a same in the predetermined cycle.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a schematic configuration of an endoscope system according to an embodiment;

FIG. 2 is an exploded perspective view illustrating a configuration of an optical unit according to the embodiment;

FIG. 3 is a sectional view illustrating a configuration of main parts of the optical unit when viewed as a cross section that an axis O in FIG. 2 passes through;

FIG. 4 is a sectional view of the optical unit when viewed as a cross section on a line IV-IV in FIG. 3;

FIG. 5 is a sectional view of the optical unit when viewed as a cross section on a line V-V in FIG. 3;

FIG. 6 is a perspective view illustrating a configuration of a movable portion according to the embodiment;

FIG. 7 is a top view of the movable portion according to the embodiment;

FIG. 8 is a block diagram illustrating a functional configuration of the endoscope system according to the embodiment;

FIG. 9 is a flowchart illustrating an outline of a drive process for an optical unit 10, the drive process being a process to be executed by the endoscope system according to the embodiment;

FIG. 10 is a timing chart illustrating an outline of the drive process for the optical unit, the drive process being the process to be executed by the endoscope system according to the embodiment;

FIG. 11 is a diagram schematically illustrating a situation where the movable portion has stopped at a normal position with respect to a fixed portion according to the embodiment;

FIG. 12 is a diagram schematically illustrating a situation where the movable portion has stopped at a near position (magnification) with respect to the fixed portion according to the embodiment;

FIG. 13 is a diagram illustrating a table representing an example of results of comparison between effective currents for cycles, each in which a driver according to the embodiment supplies a first current and a second current; and

FIG. 14 is a top view of a movable portion according to a modified example the embodiment.

### DETAILED DESCRIPTION

Embodiments according to the present disclosure will hereinafter be described in detail, together with the drawings. The present disclosure is not to be limited by the following embodiments. The drawings referred to in the following description schematically illustrate shapes, sizes, and positional relations merely to an extent that allows the present disclosure to be understood. That is, the present disclosure is not to be limited only to the shapes, sizes, and positional relations exemplified by the drawings.

### Schematic Configuration of Endoscope System

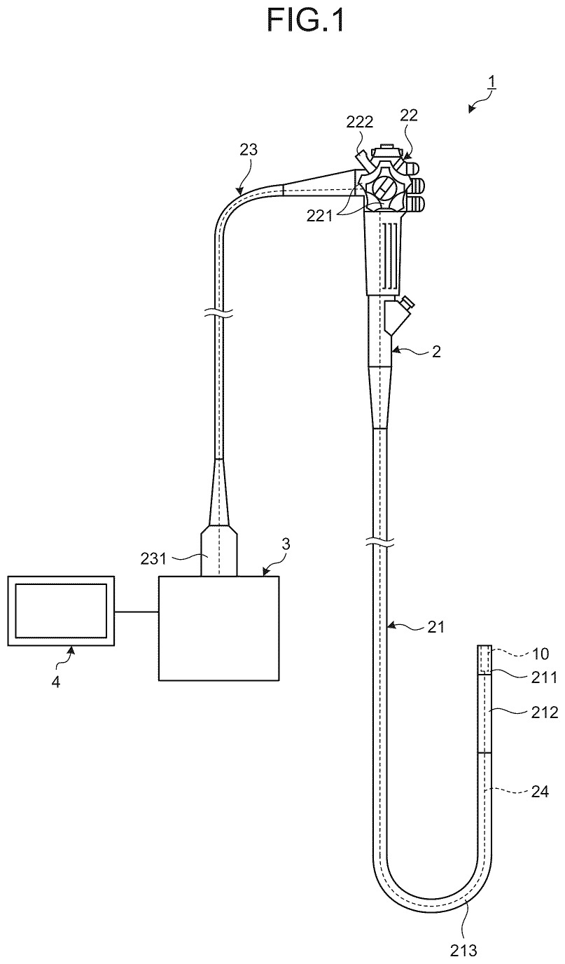

FIG. 1 is a diagram illustrating a schematic configuration of an endoscope system according to an embodiment. An endoscope system 1 illustrated in FIG. 1 includes an endoscope 2, a control device 3, and a display device 4.

The endoscope 2 is able to be introduced into a subject, such as a human body, implements imaging of a predetermined region to be observed in the subject, and generates imaging data. The subject, into which the endoscope 2 is introduced, is not necessarily a human body, and may be another living body, or an artificial object, such as a machine or a building. In other words, the endoscope 2 may be a flexible or rigid medical endoscope or an industrial endoscope. The endoscope 2 described hereinafter is a medical endoscope, for example, a nasal endoscope.

The endoscope 2 includes: an insertion unit 21 to be inserted into a subject; an operating unit 22 positioned at a proximal end of the insertion unit 21; and a universal cord 23 serving as a composite cable extending from the operating unit 22.

The insertion unit 21 has: a distal end portion 211 that is provided at a distal end of the insertion unit 21 and outputs illumination light to an object in a subject; a bending portion 212 that is provided near a proximal end of the distal end portion 211 and is bendable; and a flexible tube portion 213 that is provided near a proximal end of the bending portion 212, is connected to a distal end of the operating unit 22, and has flexibility. The distal end portion 211 has, provided therein, an imaging unit 214 that condenses light from the object and captures an object image of the object.

The imaging unit 214 has: the optical unit 10 that condenses light from an object and forms an object image; and an imaging element that receives the object image formed by the optical unit 10 and generates imaging data by photoelectric conversion of the object image. The imaging element is composed using an image sensor, such as a charge coupled device (CCD) or a complementary metal oxide semiconductor (CMOS). Detailed configurations of the optical unit 10 and the imaging element will be described later.

The operating unit 22 has: an angle operating unit 221 that controls how the bending portion 212 is bent; and a zoom operating unit 222 that outputs a focus switching signal to instruct zoom operation at the optical unit 10 described later. In the embodiment, the angle operating unit 221 is formed in a knob shape and the zoom operating unit 222 is formed in a lever shape, but without being limited to these shapes, they may each be formed in any other form and may thus be any of a volume switch, a push switch, and a toggle switch, for example.

The universal cord 23 is a member that connects the operating unit 22 and the control device 3 to each other. The endoscope 2 is connected to the control device 3 through a connector 231 provided at a proximal end portion of the universal cord 23.

A cable 24 including any of a wire, an electric line, and an optical fiber is inserted in the insertion unit 21, the operating unit 22, and the universal cord 23.

The control device 3 controls each unit included in the endoscope system 1 and integrally controls the whole endoscope system 1. The control device 3 supplies illumination light to be emitted to an object, to the endoscope 2, and outputs image data to the display device 4, the image data resulting from various kinds of image processing of imaging data generated by the imaging unit 214. The control device 3 is composed using, for example: a memory; and a processor comprising hardware, such as a central processing unit (CPU), an application specific integrated circuit (ASIC), or a field-programmable gate array (FPGA). A detailed configuration of the control device 3 will be described later.

Under control by the control device 3, the display device 4 displays a display image corresponding to image data input from the control device 3. The display device 4 is composed using, for example, a liquid crystal display, or an organic electroluminescent display.

### Configuration of Optical Unit

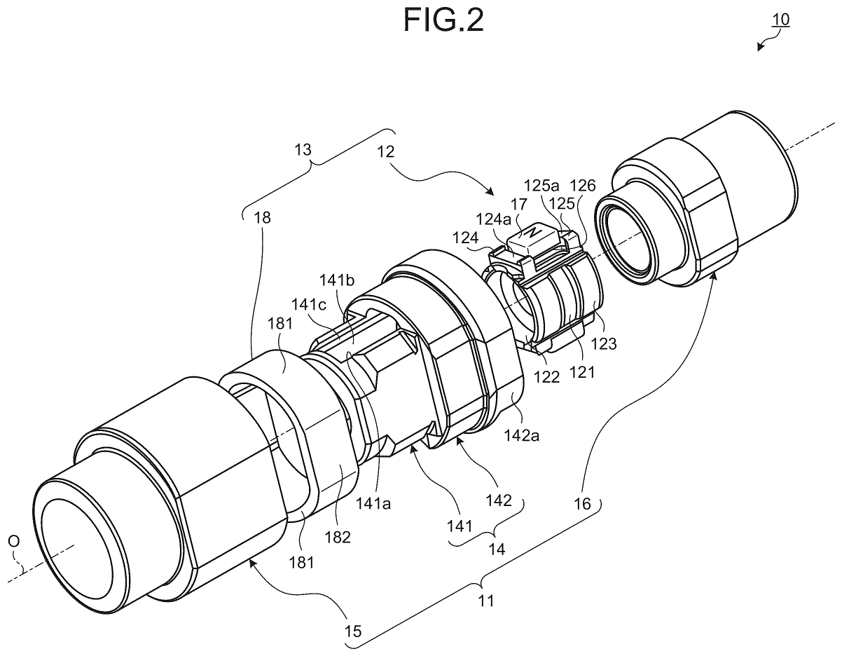

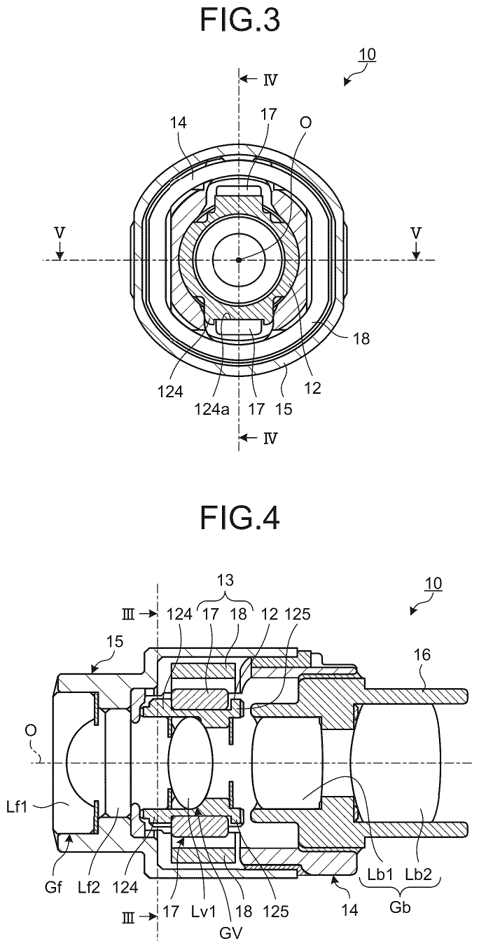

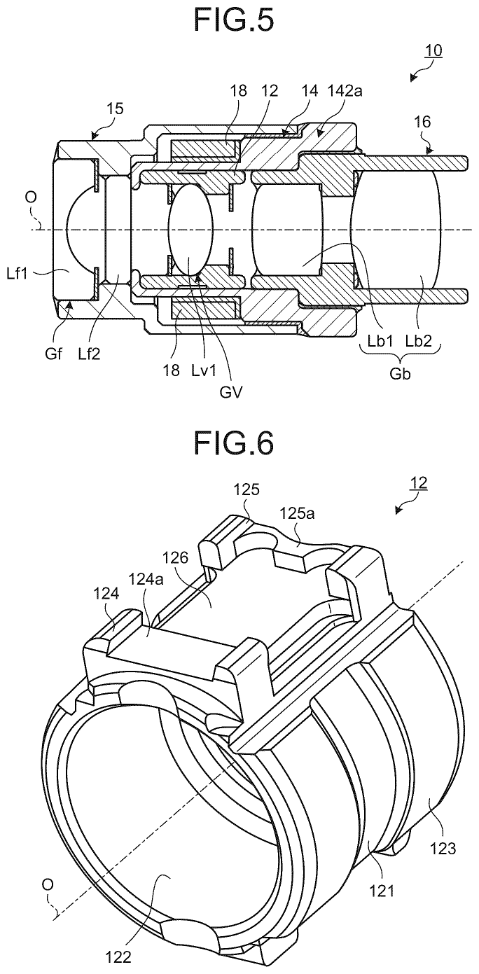

The detailed configuration of the optical unit 10 will be described next. FIG. 2 is an exploded perspective view illustrating a configuration of an optical unit. FIG. 3 is a sectional view illustrating a configuration of main parts of the optical unit 10 when viewed as a cross section that an axis O in FIG. 2 passes through. FIG. 4 is a sectional view of the optical unit 10 when viewed as a cross section on a line IV-IV in FIG. 3. FIG. 5 is a sectional view of the optical unit 10 when viewed as a cross section on a line V-V in FIG. 3. In an example described hereinafter, the axis O passing through the optical unit 10 coincides with an optical axis of the optical unit 10. Along the axis O, a direction opposite to a direction toward an object will hereinafter be referred to as a direction toward an image. That is, in the optical unit 10 illustrated in FIG. 2, a leftward direction is an object direction toward the object and a rightward direction is an image direction toward the image. A central axis of each member may also be referred to as the axis O. This is because the central axis of each member coincides with the axis O upon assembly.

The optical unit 10 illustrated in FIG. 2 to FIG. 5 includes: a fixed portion 11 having a tubular shape; a movable portion 12 that is slidable inside the fixed portion 11; and a voice coil motor 13 that generates driving force to move the movable portion 12 with respect to the fixed portion 11 by sliding the movable portion 12 along an optical axis of a lens. In this embodiment, the voice coil motor 13 functions as a drive portion.

### Configuration of Fixed Portion

The fixed portion 11 includes: a fixed portion body 14; a front frame unit 15 that holds an object direction fixed lens group Gf that is on a side of the object with respect to a movable lens group Gv held by the movable portion 12, the front frame unit 15 having been attached to the fixed portion body 14 near the object; and a rear frame unit 16 that holds an image direction fixed lens group Gb that is on a side of the image with respect to the movable lens group Gv, the rear frame unit 16 having been attached to the fixed portion body 14 near the image.

The fixed portion body 14 includes a member having a tubular shape having the axis O at its center. The fixed portion body 14 has a first tubular portion 141 having the axis O as its central axis, and a second tubular portion 142 formed on one side of the first tubular portion 141, the one side being toward the image along the axis O. The fixed portion body 14 has 90-degree rotational symmetry relative to the axis O.

The first tubular portion 141 has a tubular shape having a diameter smaller than a diameter of an outer periphery of the second tubular portion 142. Two penetrating portions 141a that penetrate the first tubular portion 141 in a direction (radial direction) orthogonal to the axis O have been formed on an upper surface and a lower surface of the first tubular portion 141. Specifically, the two penetrating portions 141a penetrating the first tubular portion 141 in the radial direction have been formed in the first tubular portion 141, symmetrically about the axis O in a longitudinal direction of the first tubular portion 141. A radially inner surface of the first tubular portion 141 excluding the penetrating portions 141a is a cylindrical surface having a tubular shape and forms a fixed sliding surface 141b that guides and supports the movable portion 12. Furthermore, two rail portions 141c extending along the axis O have been formed on an inner peripheral surface of the first tubular portion 141. The penetrating portions 141a and the rail portions 141c have both been formed at the top and the bottom of a cross section cut along a plane orthogonal to the axis O.

The second tubular portion 142 has a stepped portion 142a formed therein, the stepped portion 142a having a stepped shape formed of an end portion protruding, the end portion being that of an outer peripheral portion of the second tubular portion 142 and being toward the image. Part of the second tubular portion 142 is accommodated in the front frame unit 15 and the stepped portion 142a comes into contact with the front frame unit 15. Furthermore, the second tubular portion 142 accommodates the rear frame unit 16 therein.

The front frame unit 15 holds the object direction fixed lens group Gf. The object direction fixed lens group Gf has plural lenses including an objective lens Lf1 (the objective lens Lf1 and a lens Lf2, herein), the plural lenses having been arranged next to each other along the axis O.

The rear frame unit 16 holds the image direction fixed lens group Gb. The image direction fixed lens group Gb has a lens Lb1 and a lens Lb2 that are next to each other along the axis O. Part of the rear frame unit 16 is accommodated in the second tubular portion 142.

### Configuration of Movable Portion

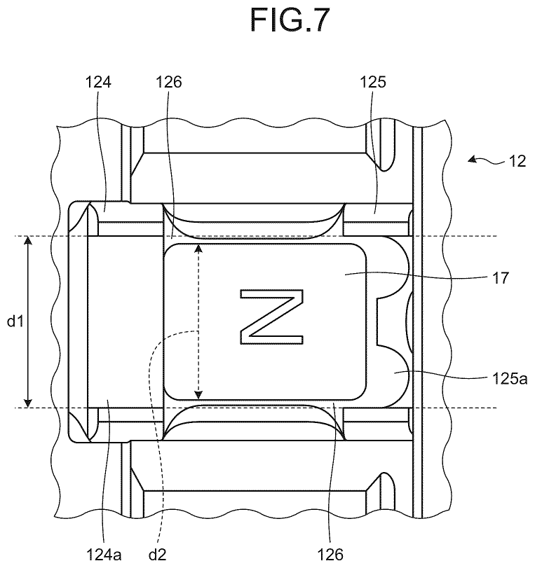

The movable portion 12 will be described next. FIG. 6 is a perspective view illustrating a configuration of the movable portion 12. FIG. 7 is a top view of the movable portion 12.

The movable portion 12 illustrated in FIG. 6 and FIG. 7 includes a tubular member having an outer peripheral portion 121 and an inner peripheral portion 122. A central axis of the movable portion 12 will hereinafter be referred to as the axis O also. This is because the central axis of the movable portion 12 and a central axis of the fixed portion body 14 coincide with each other upon assembly.

The outer peripheral portion 121 has: a tubular portion 123 having an outer peripheral surface that functions as a movable sliding surface; an object end rotation restriction portion 124 and an image end rotation restriction portion 125 that are formed on each of upper and lower surfaces of the tubular portion 123, the upper and lower surfaces being at both end portions of a length of the tubular portion 123, the length being along the axis O; and a stepped portion 126. The tubular portion 123, the object end rotation restriction portion 124, the image end rotation restriction portion 125, and the stepped portion 126 may be formed as an integral member or as separately bodied members.

The object end rotation restriction portion 124 and the image end rotation restriction portion 125 have a groove portion 124a and a groove portion 125a formed therein, the groove portions 124a and 125a each having a planar outer peripheral surface formed more radially outward than the tubular portion 123. By being slidably held by the rail portions 141c of the fixed portion body 14, the object end rotation restriction portion 124 and the image end rotation restriction portion 125 enable movement of the movable portion 12 forward and rearward along the axis O and restrict rotation of the movable portion 12 about the axis O. Deflection of the movable portion 12 upon movement of the movable portion 12 is thereby able to be prevented.

The stepped portion 126 is formed more radially outward than the tubular portion 123 and has a planar outer peripheral surface. A magnet 17 is arranged on an upper surface of the stepped portion 126. The groove portion 124a and the groove portion 125a are formed to have a width d1 of a length substantially matching a width d2 of the magnet 17, the width d1 being in a circumferential direction on a plane orthogonal to the axis O in the movable portion 12, the width d2 being in the circumferential direction on the same plane. The magnet 17 is thereby able to be arranged easily on a plane of the stepped portion 126.

The movable portion 12 holds the movable lens group Gv. Specifically, the movable portion 12 holds a movable first lens Lv1 included in the movable lens group Gv. As illustrated in FIG. 2 to FIG. 7, one side of the movable first lens Lv1 is preferably in contact with an inner peripheral side, the one side being toward the image.

The movable portion 12 configured as described above is inserted in the fixed portion body 14, with the tubular portion 123 coming in contact with the fixed sliding surface 141b. The movable portion 12 is formed using a material, such as stainless steel, aluminum, or resin, for example.

### Configuration of Voice Coil Motor

A configuration of the voice coil motor 13 will be described next. As illustrated in FIG. 2 to FIG. 7, the voice coil motor 13 includes a coil 18 arranged on the fixed portion body 14 of the fixed portion 11, and the magnet 17 arranged on the movable portion 12 to face the coil 18.

The coil 18 is arranged in a state of being wound around an outer periphery of the first tubular portion 141 of the fixed portion body 14. A coil 18 that has been coiled up beforehand may be arranged there later instead. The coil 18 includes cylindrical portions 181 facing the penetrating portions 141a of the fixed portion body 114, and planar portions 182 facing lateral surfaces of the fixed portion body 14. The coil 18 is formed of the two cylindrical portions 181 and the two planar portions 182 alternately arranged in a cross section thereof orthogonal to the axis O.

As illustrated in FIG. 2 to FIG. 7, the magnet 17 is arranged at the stepped portion 126 of the movable portion 12 along the axis O inside the cylindrical portions 181 of the coil 18, so as to face the cylindrical portions 181.

A stable magnetic field is formed in the voice coil motor 13 configured as described above and deflection of the movable portion 12 that moves relatively to the fixed portion 11 is able to be minimized. In the embodiment, the magnet 17 (a first magnet and a second magnet) is arranged every 180 degrees about the axis O but the magnet 17 may be arranged at any other angular intervals.

### Functional Configuration of Endoscope System

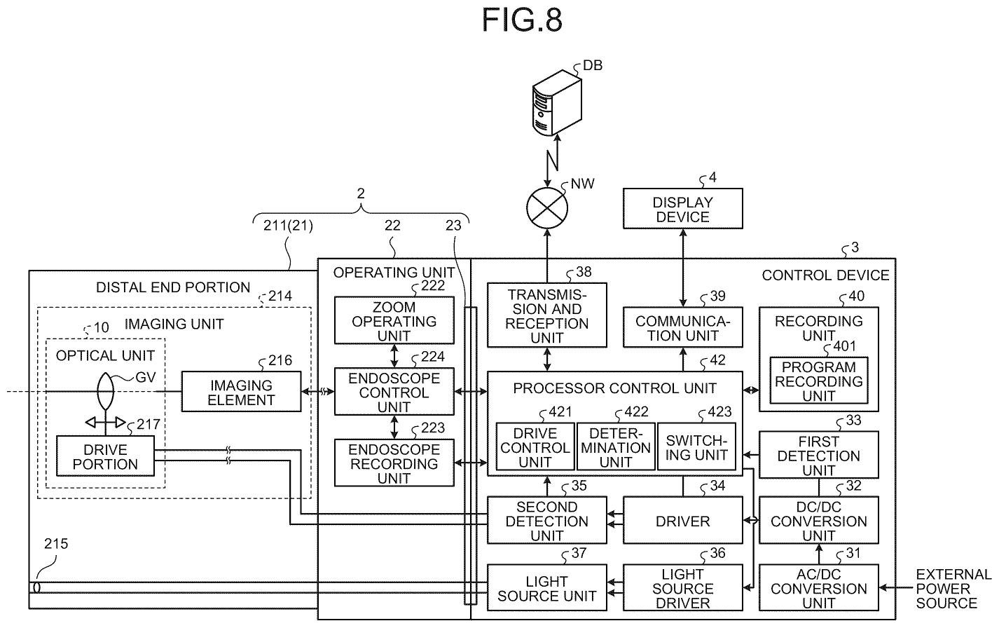

A functional configuration of the endoscope system 1 will be described next. FIG. 8 is a block diagram illustrating the functional configuration of the endoscope system 1. As illustrated in FIG. 8, the endoscope system 1 includes the endoscope 2, the control device 3, and the display device 4.

### Functional Configuration of Endoscope

A functional configuration of the endoscope 2 will be described first. The endoscope 2 includes at least the distal end portion 211, the operating unit 22, and the universal cord 23.

The distal end portion 211 has, provided therein, the imaging unit 214 and an illumination lens 215. The imaging unit 214 has an imaging element 216 and a drive portion 217.

Under control by the control device 3 described later, the imaging element 216 generates imaging data on an object image formed by a lens group of the optical unit 10 described above, and outputs the generated imaging data to the control device 3.

The drive portion 217 includes the voice coil motor 13 of the optical unit 10 described above, and under control by the control device 3 described later, moves the movable portion 12 and the movable lens group Gv held by the movable portion 12, along the axis O.

The illumination lens 215 outputs illumination light supplied from the control device 3 described later via, for example, a light guide of the universal cord 23, to an object. The illumination lens 215 is composed using one or plural lenses.

The operating unit 22 has at least: the above described zoom operating unit 222; an endoscope control unit 224 that controls driving of the drive portion 217 and the imaging element 216; and an endoscope recording unit 225 that records therein various types of information related to the endoscope 2.

The endoscope control unit 224 is composed using a memory, and a processor having hardware, such as a CPU, an FPGA, or an ASIC. The endoscope control unit 224 controls the imaging element 216 and the drive portion 217, according to a control signal transmitted from the control device 3 and a focus switching signal from the zoom operating unit 222. The endoscope control unit 224 subjects imaging data input from the imaging element 216 to predetermined image processing and outputs a result thereof to the control device 3.

An endoscope recording unit 223 is composed using a flash memory, a ROM, and a RAM, and records therein various types of information related to the endoscope 2. Specifically, type information on the endoscope 2, identification information on the endoscope 2, and a manufacturing date of the endoscope 2, for example, are recorded in the endoscope recording unit 223.

### Functional Configuration of Control Device

A functional configuration of the control device 3 will be described next. The control device 3 includes an AC/DC conversion unit 31, a DC/DC conversion unit 32, a first detection unit 33, a driver 34, a second detection unit 35, a light source driver 36, a light source unit 37, a transmission and reception unit 38, a communication unit 39, a recording unit 40, and a processor control unit 42.

The AC/DC conversion unit 31 converts an alternating current supplied from an external power source into a direct current and outputs the direct current to the DC/DC conversion unit 32. The AC/DC conversion unit 31 is composed using a switching regulator, for example.

The DC/DC conversion unit 32 converts the direct current input from the AC/DC conversion unit 31 into a predetermined voltage value, and outputs the predetermined voltage value to each unit included in the control device 3, for example, the driver 34, the light source driver 36, and the processor control unit 42. The DC/DC conversion unit 32 is composed using a linear regulator, or a switching regulator, for example.

The first detection unit 33 is electrically connected in parallel and in series with wiring electrically connecting between the DC/DC conversion unit 32 and the processor control unit 42. The first detection unit 33 detects a current value and a voltage value output by the DC/DC conversion unit 32 and outputs these detection results to the processor control unit 42. The first detection unit 33 is composed using a voltage sensor and a current sensor, for example.

Under control by the processor control unit 42, the driver 34 supplies a predetermined current for driving the drive portion 217 of the endoscope 2 by using the direct current that has been adjusted to the predetermined voltage value and input from the DC/DC conversion unit 32. The driver 34 is composed using, for example, an H-bridge circuit.

The second detection unit 35 is electrically connected in series and in parallel with wiring that electrically connects between the driver 34 and the drive portion 217. The second detection unit 35 detects a current value and a voltage value supplied by the driver 34 and outputs these detection results to the processor control unit 42. The second detection unit 35 is composed using, for example, a current sensor and a voltage sensor.

Under control by the processor control unit 42, the light source driver 36 supplies a current for causing the light source unit 37 to emit light, by using the direct current input from the DC/DC conversion unit 32.

On the basis of the direct current supplied from the light source driver 36, the light source unit 37 generates white light and supplies illumination light to be emitted to an object, to the distal end portion 211 of the endoscope 2. The light source unit 37 is composed using, for example, a white light emitting diode (LED).

Under control by the processor control unit 42, the transmission and reception unit 38 performs communication with an external database server DB via a network NW, transmits, for example, imaging data generated by the endoscope 2 to the external database server DB, and receives various types of information from the external database server DB. The transmission and reception unit 38 is composed using, for example, a communication module conforming to Wi-Fi (registered trademark). The network NW may include, for example, an internet line network and/or a mobile phone line network.

The communication unit 39 outputs image data to the display device 4, under control by the processor control unit 42. The communication unit 39 is composed using, for example, a communication module, such as High-Definition Multimedia Interface (HDMI) (registered trademark).

The recording unit 40 records therein various types of information related to the endoscope system 1. The recording unit 40 has a program recording unit 401 that records therein various programs executed by the endoscope system 1. The recording unit 40 is composed using, for example, a random access memory (RAN), a read only memory (ROM), a hard disk drive (HDD), and a solid state drive (SSD).

The processor control unit 42 integrally controls the units included in the endoscope system 1. The processor control unit 42 is composed using a memory, and a processor having hardware, such as a CPU, an FPGA, and an ASIC. The processor control unit 42 reads the programs recorded in the program recording unit 401 into a work area of the memory, executes the programs read, and controls each unit through execution of the programs by the processor, and hardware and software thereby cooperate with each other and implement functional modules matching predetermined purposes. Specifically, the processor control unit 42 has, as the functional modules, a drive control unit 421, a determination unit 422, and a switching unit 423. In the embodiment, the optical unit 10, the driver 34, and the processor control unit 42 function as a drive device.

The drive control unit 421 causes the driver 34 to supply a first current or a second current to the drive portion 217. Furthermore, the drive control unit 421 causes the driver 34 to supply current such that the second current is supplied within a predetermined time period. In the embodiment, the drive control unit 421 functions as a controller.

The determination unit 422 determines whether or not a predetermined time period that is a second supply time period has elapsed since the driver 34 started supplying the first current to the drive portion 217.

The switching unit 423 changes the direction of a current that the driver 34 supplies to the drive portion 217. Specifically, when the determination unit 422 determines that a focus switching signal to change the focus has been input from the zoom operating unit 222 in a time period, in which the driver 34 is supplying the first current to the drive portion 217, the switching unit 423 switches the direction of the current supplied by the driver 34 to the drive portion 217 from a positive (+) to a negative (−) or from the negative (−) to the positive (+).

### Drive Process for Optical Unit

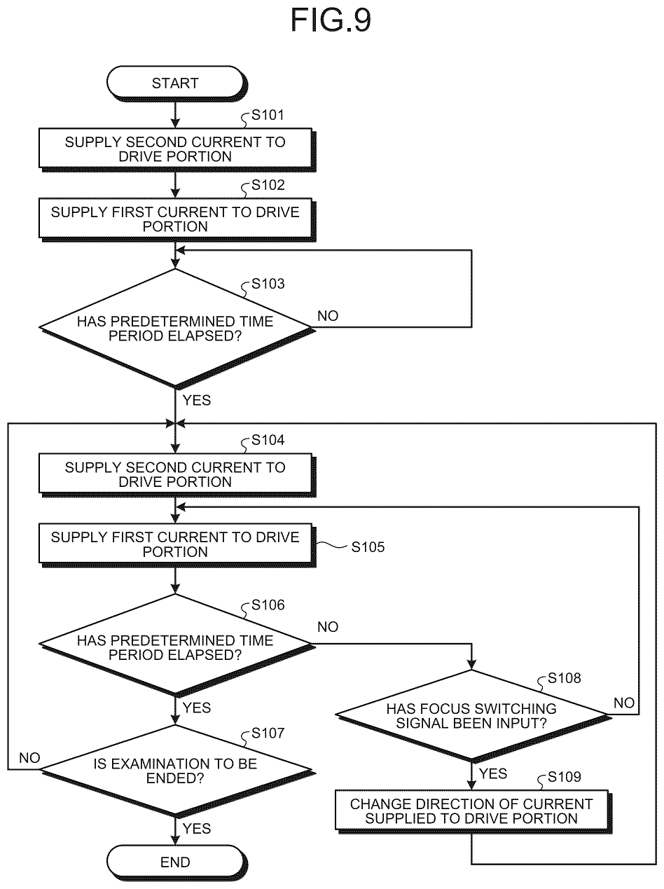

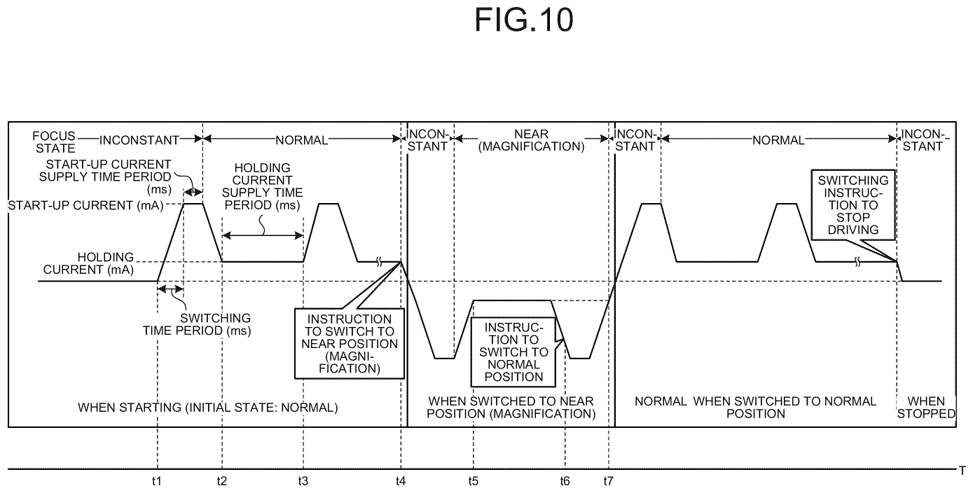

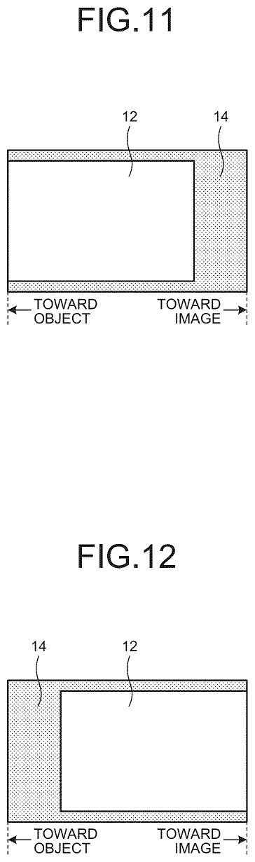

An outline of a drive process executed by the endoscope system 1, for the optical unit 10 will be described next. FIG. 9 is a flowchart illustrating the outline of the drive process executed by the endoscope system 1, for the optical unit 10. FIG. 10 is a timing chart illustrating the outline of the drive process executed by the endoscope system 1, for the optical unit 10. FIG. 11 is a diagram schematically illustrating a situation where the movable portion 12 has stopped at a normal position with respect to the fixed portion 11. FIG. 12 is a diagram schematically illustrating a situation where the movable portion 12 has stopped at a near position (magnification) with respect to the fixed portion 11. In FIG. 10, the horizontal axis represents time and the vertical axis represents current.

As illustrated in FIG. 9, firstly, the drive control unit 421 causes the driver 34 to supply the second current to the drive portion 217 (Step S101). In this case, as illustrated in FIG. 10, immediately after starting of the endoscope 2 (time t1), the drive control unit 421 causes the driver 34 to supply the second current in a first supply time period including a switching time period and a start-up time period. The drive control unit 421 causes the driver 34 to supply current to the drive portion 217 with a gradient such that the second current is supplied within the switching time period. As illustrated in FIG. 11, the movable portion 12 thereby moves in the fixed portion 11 near the object and butts against the fixed portion 11 and the drive portion 217 thus enables prevention of overshooting and undershooting due to a rapid current change.

Thereafter, the drive control unit 421 causes the driver 34 to supply the first current that is a holding current for holding the movable portion 12 at the fixed portion 11, to the drive portion 217 in a second supply time period (Step S102). In this case, as illustrated in FIG. 10, the drive control unit 421 causes the driver 34 to supply the first current to the drive portion 217 in the second supply time period (time t2). The movable portion 12 is thereby held at a position (hereinafter, simply referred to as a “first position”) where the movable portion 12 butts against the fixed portion 11 near the object.

Subsequently, the determination unit 422 determines whether or not the predetermined time period that is the second supply time period has elapsed since the driver 34 started supplying the first current to the drive portion 217 (Step S103). In a case where the determination unit 422 determines that the predetermined time period has elapsed (Step S103: Yes), the endoscope system 1 proceeds to Step S104 described later. On the contrary, in a case where the determination unit 422 determines that the predetermined time period has not elapsed (Step S103: No), the endoscope system 1 waits until the predetermined time period elapses.

At Step S104, the drive control unit 421 causes the driver 34 to supply the second current to the drive portion 217. In this case, as illustrated in FIG. 10, the drive control unit 421 causes the driver 34 to supply the second current to the drive portion 217 in the first supply time period (time t3). As illustrated in FIG. 11, the movable portion 12 thereby butts against the fixed portion 11 near the object in the fixed portion 11. As a result, even if the movable portion 12 is displaced by an external disturbance in a case where the driver 34 is supplying the first current to the drive portion 217, the movable portion 12 is able to be held at the first position, an effective current is able to be maintained less than a predetermined value on a predetermined cycle, and the optical unit 10 is thus able to be prevented from being heated.

Subsequently, the drive control unit 421 causes the driver 34 to supply the first current to the drive portion 217 in the second supply time period (Step S105). The movable portion 12 is thereby able to be maintained at the first position in the fixed portion 11.

Thereafter, the determination unit 422 determines whether or not the predetermined time period that is the second supply time period has elapsed since the driver 34 started supplying the first current to the drive portion 217 (Step S106). In a case where the determination unit 422 determines that the predetermined time period has elapsed (Step S106: Yes), the endoscope system 1 proceeds to Step S107 described later. On the contrary, in a case where the determination unit 422 determines that the predetermined time period has not elapsed (Step S106: No), the endoscope system 1 proceeds to Step S108 described later.

At Step S107, the determination unit 422 determines whether or not an end signal to instruct ending of an examination has been input from the operating unit 22. In a case where the determination unit 422 determines that the end signal to instruct the ending of the examination has been input from the operating unit 22 (Step S107: Yes), the endoscope system 1 ends this process. On the contrary, in a case where the determination unit 422 determines that the end signal to instruct the ending of the examination has not been input from the operating unit 22 (Step S107: No), the endoscope system 1 proceeds to Step S104 described above.

At Step S108, the determination unit 422 determines whether or not a focus switching signal to change the focus has been input from the zoom operating unit 222. Specifically, as illustrated in FIG. 10, the determination unit 422 determines whether or not a focus switching signal to change the focus has been input from the zoom operating unit 222 in the second supply time period, in which the driver 34 is supplying the first current to the drive portion 217. In a case where the determination unit 422 determines that a focus switching signal to change the focus has been input from the zoom operating unit 222 (Step S108: Yes), the endoscope system 1 proceeds to Step S109 described later. On the contrary, in a case where the determination unit 422 determines that a focus switching signal to change the focus has not been input from the zoom operating unit 222 (Step S108: No), the endoscope system 1 returns to Step S105 described above.

At Step S109, the switching unit 423 changes the direction of the current that the driver 34 supplies to the drive portion 217. After Step S109, the endoscope system 1 returns to Step S104 described above. In this case, as illustrated in FIG. 10, when the determination unit 422 determines that a focus switching signal to change the focus has been input from the zoom operating unit 222 in a time period, in which the driver 34 is supplying the first current to the drive portion 217 (time t4), the switching unit 423 changes the direction of the current supplied by the driver 34 to the drive portion 217 by inverting the direction to the negative. As illustrated in FIG. 10, the drive control unit 421 thereby causes the driver 34 to supply a negative second current in the first application time period including the switching time period and the start-up time period (time t4). In this case, the drive control unit 421 causes the driver 34 to supply current to the drive portion 217 with a gradient such that the second current is supplied within the switching time period. As a result, as illustrated in FIG. 12, the movable portion 12 butts against the fixed portion 11 at a position (hereinafter, simply referred to as a “second position”) near the image, and the drive portion 217 thus enables prevention of overshooting and undershooting due to a rapid current change.

Furthermore, the drive control unit 421 causes the driver 34 to supply a negative first current that is a holding current for holding the movable portion 12 at the second position on a predetermined cycle to the drive portion 217 in the second supply time period (time t5). The movable portion 12 is thereby able to be held at the second (near) position in the fixed portion 11, the effective current is thereby able to be maintained less than the predetermined value on the predetermined cycle, and the optical unit 10 is thus able to be prevented from being heated. In this case, similarly, when the determination unit 422 determines that a focus switching signal to change the focus has been input from the zoom operating unit 222 in a time period, in which the driver 34 is supplying the negative first current to the drive portion 217, the switching unit 423 changes the direction of the current supplied by the driver 34 to the drive portion 217 by inverting the direction to the positive. As a result, the endoscope system 1 implements a process similar to Step S104 and Step S105 described above.

By contrast, when the determination unit 422 determines that a focus switching signal to change the focus has been input from the zoom operating unit 222 in a time period, in which the driver 34 is supplying the negative second current to the drive portion 217 (time t6), the switching unit 423 changes the direction of the current supplied by the driver 34 to the drive portion 217 by inverting the direction to the positive after elapse of the first supply time period for supply of the second current (time t7) and supplies a positive second current.

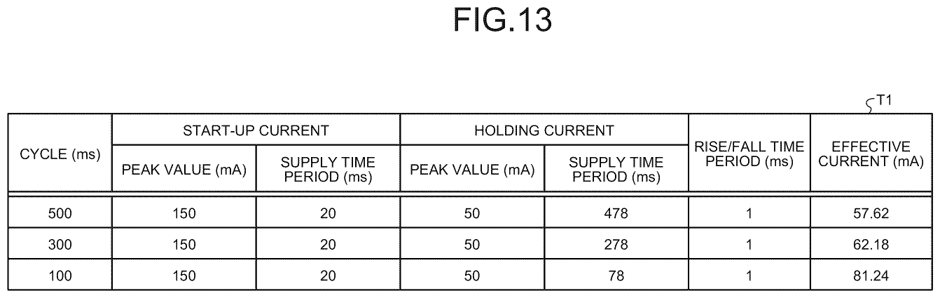

Results of comparison between effective currents for cycles, each on which the driver 34 supplies the first current and the second current will be described next. FIG. 13 is a diagram illustrating a table representing the results of the comparison between the effective currents for the cycles, each on which the driver 34 supplies the first current and the second current.

As represented by a comparison result table Ti illustrated in FIG. 13, the drive control unit 421 causes the driver 34 to repeatedly supply the second current larger than the first current on predetermined cycles of 100 to 500 ms. The predetermined cycle is preferably 300 to 500 ms and more preferably 500 ms. By causing the driver 34 to repeatedly supply the second current larger than the first current on the predetermined cycle of 500 ms, the drive control unit 421 enables the effective current to be maintained at 60 mA and thus enables prevention of generation of heat in the optical unit 10.

According to the embodiment described above, the drive control unit 421 causes the driver 34 to supply the first current to the drive portion 217 and causes the driver 34 to repeatedly supply the second current larger than the first current on the predetermined cycle, a Hall generator for detecting a position of the movable portion 12 is thus not required to be provided, and the diameter and length of the distal end portion 211 are thus able to be decreased.

Furthermore, according to the embodiment, in a case where a focus switching signal to instruct the focus to be changed has been input, the switching unit 423 changes the direction of the current supplied by the driver 34 to the drive portion 217 in causing the driver 34 to supply the first current and the second current, and the focal length is thus able to be changed with a simple configuration.

Furthermore, according to the embodiment, in a case where a focus switching signal to instruct the focus to be changed has been input, the switching unit 423 changes the direction of the current supplied by the driver 34 to the drive portion 217 in a supply time period, in which the first current is supplied by the driver 34, and the movable portion 12 is thus able to be moved smoothly.

Furthermore, according to the embodiment, the drive control unit 421 causes the driver 34 to supply the current such that the second current is supplied within the predetermined time period, and the drive portion 217 thus enables prevention of overshooting and undershooting due to a rapid current change.

Furthermore, according to the embodiment, the predetermined cycle is 100 to 500 ms, heat generation at the drive portion 217 is thus able to be minimized, and heat generation at the distal end portion 211 is thus able to be prevented.

### Modified Example

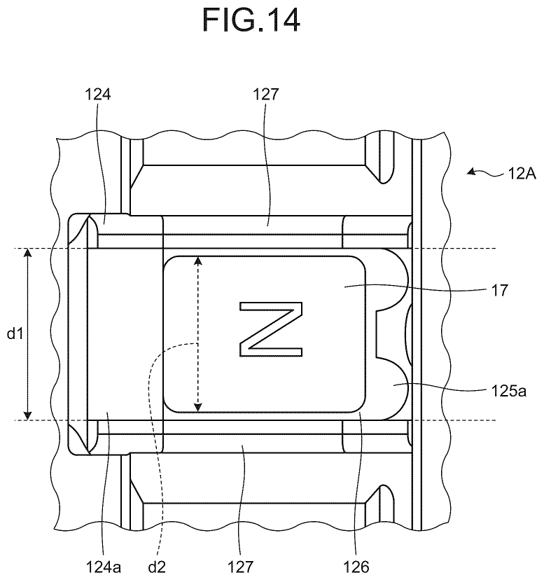

A modified example of the embodiment will be described next. FIG. 14 is a top view of a movable portion according to the modified example of the embodiment. A movable portion 12A illustrated in FIG. 14 has, in addition to the above described configuration of the movable portion 12, a coupling portion 127 that couples between the object end rotation restriction portion 124 and the image end rotation restriction portion 125. Deflection upon movement of the movable portion 12A is thereby able to be prevented.

### OTHER EMBODIMENTS

Various embodiments may be formed by combination, as appropriate, of plural components disclosed with respect to the above described endoscope system according to the embodiment. For example, some of the components described with respect to the endoscope system according to the above described embodiment may be eliminated. Furthermore, any components described with respect to the endoscope system according to the above described embodiment may be combined as appropriate.

Furthermore, the “units” and “portions” described above with respect to the endoscope system according to the embodiment of the present disclosure may be read, for example, as “means” or “circuits”. For example, a control unit may be read as a control means or a control circuit.

Furthermore, a program to be executed by the endoscope system according to the embodiment of the present disclosure may be provided as file data in an installable format or executable format, by being recorded in a computer readable recording medium, such as a CD-ROM, a flexible disk (FD), a CD-R, a digital versatile disk (DVD), a USB medium, or a flash memory.

Furthermore, a program to be executed by the endoscope system according to the embodiment of the present disclosure may be configured to be stored on a computer connected to a network, such as the Internet, and to be provided by being downloaded via the network.

In the description of the flowchart in this specification, the processing order of the steps is disclosed by use of expressions, such as “firstly”, “thereafter”, and “subsequently”, but the sequence of the process needed for implementation of the embodiment of the present disclosure is not uniquely defined by these expressions. That is, the sequence of the process in the flowchart described in this specification may be modified as far as no contradiction arises from the modification.

According to the present disclosure, an effect of enabling decrease in diameter and decrease in length is able to be achieved.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A drive device, comprising:
a fixed portion;
a movable portion that is provided slidably inside the fixed portion, the movable portion being configured to hold a lens;
a drive portion configured to cause the movable portion to slide along an optical axis of the lens with respect to the fixed portion;
a driver configured to supply a current to the drive portion; and
a controller configured to control the driver,
the controller comprising a processor comprising hardware,
the processor being configured to cause the driver to supply a first current that is small to the drive portion and cause the driver to repeatedly supply a second current larger than the first current on a predetermined cycle, and
a direction of movement of the movable portion by the first current or the second current being a same in the predetermined cycle.

2. The drive device according to claim 1, wherein the processor is configured to cause the second current to be supplied after elapse of a predetermined time period since a start of supply of the first current.

3. The drive device according to claim 1, wherein the processor is configured to cause the first current to be supplied again after supply of the second current for a predetermined time period.

4. The drive device according to claim 1, wherein a supply time period for supply of the first current is longer than a supply time period for supply of the second current.

5. The drive device according to claim 1, wherein when a focus switching signal to instruct a focus to be changed has been input, the processor is configured to cause the driver to supply the first current and the second current to the drive portion by changing a direction of a current supplied by the driver to the drive portion.

6. The drive device according to claim 5, wherein when the focus switching signal has been input, the processor is configured to change the direction of the current supplied by the driver to the drive portion in a supply time period for supply of the first current by the driver.

7. The drive device according to claim 6, wherein the processor causes the driver to supply the current such that the second current is supplied within a predetermined time period.

8. The drive device according to claim 1, wherein the predetermined cycle is 100 to 500 ms.

9. The drive device according to claim 8, wherein the predetermined cycle is 300 to 500 ms.

10. The drive device according to claim 9, wherein the predetermined cycle is 500 ms.

11. The drive device according to claim 1, wherein the drive portion comprises a voice coil motor including a magnet and a coil.

12. The drive device according to claim 11, wherein the magnet includes a first magnet and a second magnet that are each provided on a different outer peripheral surface of the movable portion.

13. An endoscope system, comprising:
an endoscope including an optical unit, the endoscope being configured to be inserted inside a subject to observe an interior of the subject; and
a control device to which the endoscope is detachably connected, the control device being configured to control driving of the optical unit,
the optical unit comprising:
a fixed portion;
a movable portion that is provided slidably inside the fixed portion, the movable portion being configured to hold a lens; and
a drive portion configured to cause the movable portion to slide along an optical axis of the lens with respect to the fixed portion,
the control device comprises:
a driver configured to supply a current to the drive portion; and
a controller configured to control the driver,
the controller comprising a processor comprising hardware,
the processor being configured to cause the driver to supply a first current that is small to the drive portion and cause the driver to repeatedly supply a second current larger than the first current on a predetermined cycle, and
a direction of movement of the movable portion by the first current or the second current being a same in the predetermined cycle.

14. The endoscope system according to claim 13, wherein the predetermined cycle is 100 to 500 ms.

15. The endoscope system according to claim 14, wherein the predetermined cycle is 300 to 500 ms.

16. The endoscope system according to claim 15, wherein the predetermined cycle is 500 ms.

17. A drive method executed by a drive device comprising: a fixed portion; a movable portion that is provided slidably inside the fixed portion, the movable portion being configured to hold a lens; a drive portion configured to cause the movable portion to slide along an optical axis of the lens with respect to the fixed portion; and a driver configured to supply a current to the drive portion, the drive method including:
causing the driver to supply a first current that is small to the drive portion and causing the driver to repeatedly supply a second current larger than the first current on a predetermined cycle, and
a direction of movement of the movable portion by the first current or the second current being a same in the predetermined cycle.

18. The drive method according to claim 17, wherein the predetermined cycle is 100 to 500 ms.

19. The drive method according to claim 18, wherein the predetermined cycle is 300 to 500 ms.

20. The drive method according to claim 19, wherein the predetermined cycle is 500 ms.
